(19) United States
(12) Patent Application Publication
(10) Pub. No.: US 2008/0221517 A1
(43) Pub. Date: Sep. 11, 2008

(54) Fixed-Dose Syringe with Limited Aspiration

(76) Inventors: Thomas J. Shaw, Frisco, TX (US); Gary Wood, Frisco, TX (US); Mark Small, Leonard, TX (US)

Correspondence Address: Locke Lord Bissell & Liddell LLP;Attn: Michael Ritchie, Docketing, 2200 Ross Avenue, Suite # 2200, DALLAS, TX, 75201-6776, US

(21) Appl. No.: 12/124,506

(22) Filed: May 21, 2008

Related U.S. Application Data
(63) Continuation of application No. 10/969,128, filed on Oct. 18, 2004.

(51) Int. Cl. A61M 5/50; A61M 5/178; A61M 5/315

(52) U.S. Cl. 604/110; 604/222; 604/208

(57) ABSTRACT

A syringe configured with a limited maximum usable capacity. The syringe of the invention desirably has a retractable needle to prevent reuse. In the preferred embodiment, a dose-limiting structure includes a stop-ring member on the head of the plunger that abuts a constriction in the housing when the plunger is moved away from the needle to prevent the further rearward movement of the plunger. Preferably, the syringe of the invention is configured such that a user is tactilely signaled when the plunger has reached a position corresponding to a nominal fixed-dose. If the user attempts to force the stop-ring member beyond the constriction, the plunger seal is stripped off or removed from the plunger head and the syringe rendered inoperable. The features of the invention can also be applied to a nonretracting syringe.

### BACKGROUND OF THE INVENTION

1. Field of the Invention

This invention relates to a medical device and more particularly to a fixed-dose syringe that is designed to limit the amount of fluid drawn into the syringe to a predetermined maximum quantity.

2. Description of Related Art

Diseases, such as HIV and hepatitis, continue to spread in less developed countries at an alarming rate. A major cause of this proliferation is the reuse of syringes in mass immunization programs. Mass immunization programs, which are set up in third world countries for the purpose of preventing the transmission of infectious diseases, have vaccinators who routinely use the same needle for consecutive vaccines in order to save time and money. For example, while the standard dosage of vaccines is 0.5 ml per person, during these mass immunization programs it is often the case that a 3 cc syringe is filled with 3 ml of vaccine and used to deliver 0.5 ml of vaccine to six people.

The gravity of the threat posed by AIDS and hepatitis, and the fact that the spread of the dreaded diseases is through reuse of needles by mass immunization programs, has grabbed the attention of many. As a result, the syringe art has disclosed several autodisabling syringes (syringes designed for one-time use) that are rendered inoperable after injection of a predetermined maximum dose, reducing the risk of the transmission of blood-borne pathogens. Such devices include, for example, U.S. Pat. Nos. 4,946,441; 4,961,728; 4,973,310; 5,000,737; 5,562,623; and 6,283,941. Many of the prior art devices, however, contain numerous complicated parts that substantially increase manufacturing costs. Further, the prior art has not produced a fixed-dose syringe for mass production and assembly which is simple, reliable, cost effective, and easy to use, enabling an injection to be performed with quickness, ease, precision and uniformity.

A fixed-dose syringe is therefore needed that can be manufactured economically and reliably at high speed, that will fully protect the user and others from accidental needle sticks and exposure to blood-borne pathogens, and that is easier, faster and more accurate to use during mass immunization programs because of a limited maximum usable capacity. These and other advantages are provided by the invention disclosed below.

### SUMMARY OF THE INVENTION

A fixed-dose syringe is disclosed herein that provides significant advantages over the prior art. If used properly, the syringe of the present invention is rendered unusable following completion of an injection by the use of a retractable needle. This feature of the syringe serves to discourage the reuse of needles, especially in mass immunization programs, and lessens the likelihood of disease transmission in such programs. Further, the syringe is designed to limit the amount of fluid drawn. The invention includes a dose-limiting structure that renders the syringe easier, faster and more accurate to use, especially during mass immunization programs. It should be understood, however, that the dose-limiting advantages of the invention could also be applied to a nonretracting syringe.

The fixed-dose syringe of the invention comprises an elongated hollow syringe housing preferably having a retraction mechanism mounted in the front end of the housing. The retraction mechanism desirably comprises a needle holder, separable retaining member and spring. A retractable needle biased for retraction in a rearward axial direction is fixed in the needle holder. Disposed in the hollow housing is a plunger comprising a plunger seal in sliding sealed contact with the interior wall of the housing. The plunger also comprises an end cap for applying thumb force and a retraction cavity that receives the retracted needle when the retraction mechanism is actuated by forward movement of the plunger after fluid has been discharged during an injection.

In a preferred embodiment of the invention, the dose-limiting structure includes an inwardly directed projection on the inside wall of the housing that is positioned behind a specific point corresponding to a maximum intended dose. The dose-limiting structure also includes a stop-ring member that is disposed on the plunger rearwardly of and adjacent to the plunger seal. During aspiration, the approximate nominal fixed dose is drawn when the stop-ring member contacts the inwardly directed projection, limiting further withdrawal of the plunger. Under normal pressure, the plunger head cannot move beyond the projection. If, however, the user attempts to force the plunger head rearwardly beyond the projection, the stop-ring member will not move beyond the projection without stripping or removing the plunger seal off the plunger, preventing the aspiration of additional fluid and preventing the reuse of the syringe even if the needle is not retracted.

### BRIEF DESCRIPTION OF THE DRAWINGS

The apparatus of the invention is further described and explained in relation to the following figures of the drawings wherein:

FIG. 1 is an elevation view of the exterior of the syringe in the pre-use position;

FIG. 2 is a longitudinal cross-sectional view taken along line 2-2 of FIG. 1;

FIG. 3 is an enlarged longitudinal cross-sectional view taken along line 3-3 of FIG. 1 showing the plunger withdrawn to a position corresponding to a nominal fixed-dose;

FIG. 4 is a longitudinal cross-sectional view, partially broken away, taken from FIG. 3 and showing the plunger head withdrawn rearwardly to a point where the plunger seal is removed from the plunger and the stop-ring member is still disposed on the plunger; and

FIG. 5 is a detail view taken from FIG. 3 showing the plunger with the stop-ring member disposed adjacent to and rearward of the plunger seal, and contacting the dose-limiting projection inside the housing wall.

Like reference numerals are used to describe like parts in all figures of the drawings.

### DESCRIPTION OF THE PREFERRED EMBODIMENTS

The structure and operation of the basic syringe and retraction mechanism as used in the present invention are disclosed, for example, in U.S. Pat. Nos. 5,385,551; 5,578,011; 5,632,733; 6,015,438; and 6,090,077, which are herein incorporated by reference. The present invention further modifies the syringe, as disclosed in those patents, to control the amount of fluid drawn into the syringe. Although the drawings depict a 1 cc syringe modified to administer a maximum dosage of 0.5 ml/cc, it should be understood that the invention is not limited to a particular dosage or size of syringe. For example, the dosage can be restricted to 1.0 ml/cc using a 3 cc syringe.

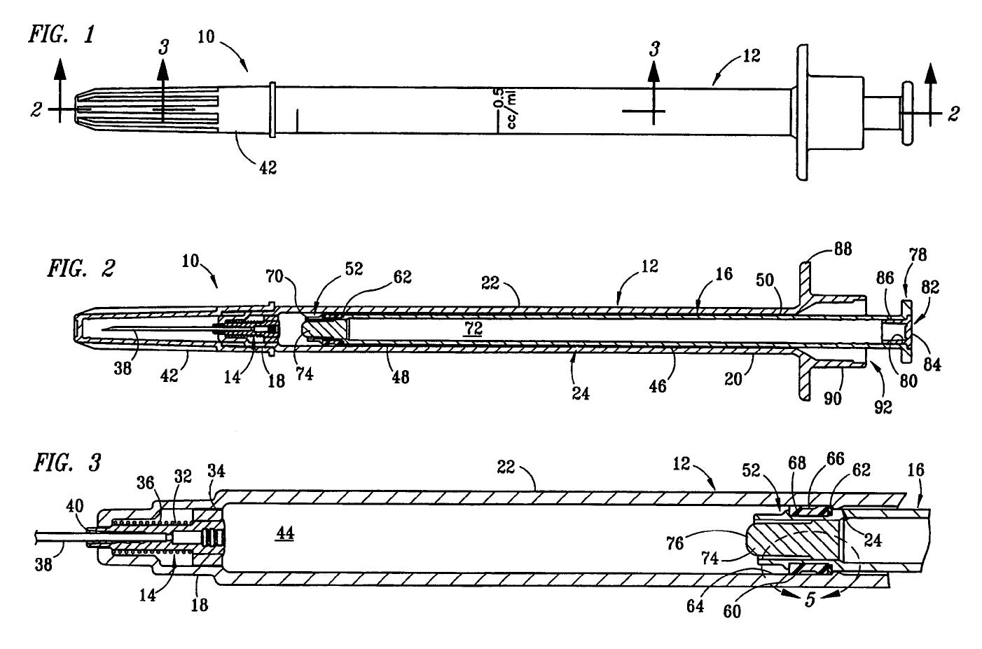

Referring to FIGS. 1 and 2, fixed-dose syringe 10 preferably comprises tubular housing 12, retraction mechanism 14 and plunger 16. Housing 12 comprises a front end portion 18 and an open back end portion 20 with a longitudinally extending wall 22 therebetween. Housing 12 is preferably molded from a substantially transparent polymeric resin such as polypropylene and may be marked with the customary volume graduations on its exterior or may only include the graduation marking for the preferred predetermined dose, which is shown in FIG. 1 as “0.5 cc/ml.”

As best seen in FIG. 5, preferably wall 22 has an inwardly directed projection 24 with chamfered sides 26, 28 and flat 30 inside syringe housing 12. Projection 24 desirably reduces the inside diameter of housing 12 by approximately 0.007 inch on each side and is an annular constriction. Those of skill in the art upon reading this disclosure will appreciate that the constriction can also be configured in other ways. For example, the constriction can be configured as circumferentially spaced projections. Projection 24 is desirably formed as an integral part of wall 22 during the molding process, but projection 24 can be formed in or inserted into housing 12 in other ways known to those of skill in the art upon reading this disclosure. Projection 24 is spaced a set distance from front end portion 18 of housing 12 so that a user can draw a predetermined maximum intended dose.

The preferred chamfer angle of side 26 is between about 15 degrees and about 45 degrees, and is more preferably about 30 degrees. The chamfer angle of side 28 is preferably between about 5 degrees to about 10 degrees, and more preferably about 10 degrees. The significance of these angles is discussed below in relation to the operation of the device.

As shown in FIGS. 2 and 3, retraction mechanism 14 is mounted within front end portion 18 of housing 12 and preferably comprises an elongated needle holder 32, separable retaining member 34, spring 36 and needle 38. Needle 38, which is preferably fixed in needle holder 32 by means of an adhesive 40, is extended in the unretracted position through front end portion 18 and covered with a removable protective cap 42. Separable retaining member 34 is preferably a ring-like structure separably and frictionally engaged with needle holder 32 to hold needle 38 against the retraction force applied by compressed spring 36.

Referring to FIGS. 2, 3 and 4, plunger 16 preferably comprises substantially cylindrical sidewall 46 with a lower end portion 48 and an upper end portion 50. Lower end portion 48 of plunger 16 comprises a head 52 with a reduced diameter portion 54 flanked by raised annular shoulders 56, 58. An elastomeric plunger seal 60 and a stop-ring member 62 are mounted on the reduced diameter portion 54 in between first and second shoulders 56, 58. Seal 60 is compressed between first shoulder 56 and stop-ring member 62, is in slidable sealed contact with the interior wall 64 of housing 12, and has channel 66 along outer wall 68. Stop-ring member 62 is compressed between plunger seal 60 and second shoulder 58, and can be configured as a circular part, split circular part, or any other similar configuration. In the preferred embodiment, stop-ring member 62 is made of a suitable medical grade polymeric material that is less compressible than plunger seal 60. One such preferred polymeric material is an acetal resin marketed under the trademark DELRIN by DuPont. Stop-ring member 62 could also be made of a suitable medical grade metal, such as stainless steel, in which case stop-ring member 62 can be made as a split ring if needed so that it can be installed over annular shoulder 56 during assembly of syringe 10. Stop-ring member 62 could also be made of a ceramic material.

The inner diameter of stop-ring member 62 is preferably sized to receive plunger head 52 therethrough with a slight gap sufficient to allow stop-ring member 62 to move freely on plunger head 52 when plunger seal 60 is not mounted on reduced diameter portion 54 of plunger head 52. The inner diameter of stop-ring member 62, however, is not large enough for stop-ring member 62 to move beyond first and second shoulders 56, 58 without sufficient deformation. The outside diameter of stop-ring member 62 is preferably sized to allow stop-ring member 62 to slide inside housing 12 in fluid chamber 44 when plunger 16 is installed in syringe 10 with plunger seal 60, but is large enough to restrict the movement of ring 62 past projection 24 during aspiration, as described below.

Plunger head 52 also has a tip portion 70 forming an opening into retraction cavity 72. Removable plug member 74 is preferably held in the opening of retraction cavity 72 by frictional engagement and cooperates with head 52 to seal the opening of retraction cavity 72. Removable plug member 74 also has a front portion 76 extending beyond tip 70. Retraction cavity 72 at least partially retains retracted needle 36, needle holder 32, plug member 74 and spring 36 upon retraction.

Upper end portion 50 of plunger 16 comprises an end cap 78 for depression of the plunger by the thumb. End cap 78 comprises a circular end wall with an opening 80 for closure 82. Closure 82 has a head 84 that is connected to a longitudinally extending annular skirt 86. Head 84 of closure 82 snuggly fits into opening 80 and effectively seals opening 80 so that fluid particles cannot escape. Annular skirt 86 frictionally engages the interior wall of plunger 16. A vent (not shown) can be disposed in end cap 78 for the venting of retraction cavity 72; however, the vent can also be located within the wall of plunger 16.

Back end portion 20 of housing 12 comprises laterally extending wings 88 and collar 90. Laterally extending wings 88 operate in conjunction with end cap 78 to allow one handed operation of the syringe. Collar 90 extends behind laterally extending wings 88 and comprises opening 92, which closely receives the outer periphery of end cap 78 when plunger 16 is depressed to the retracted position. After retraction has occurred, plunger 16 can no longer be grasped because end cap 78 is neatly tucked into opening 92.

Turning now to the operation of syringe 10, FIGS. 1 and 2 show the syringe in the ready-to-fill position. To draw fluid into fluid chamber 44, needle 38 is inserted into a liquid container, such as a vial, and head 52 of plunger 16 is pulled toward open back end portion 20 of housing 12 until stop-ring member 62 contacts inwardly directed projection 24. The user will feel contact between stop-ring member 62 and inwardly directed projection 24. At this point, a preferred limited maximum usable capacity of the syringe has been met. FIG. 3 depicts the plunger approaching the position corresponding to the nominal capacity of syringe 10. As used herein, “nominal capacity” shall mean the intended fixed dosage set by the manufacturer. As used herein, the term “limited maximum usable capacity” of a syringe shall mean the capacity sufficient to aspirate the intended nominal capacity. For example, as one skilled in the art will appreciate, when stop-ring member 62 contacts projection 24, preferably about 10 percent to about 20 percent of fluid in excess of the intended fixed-dose is drawn into syringe 10 so that the user can remove air bubbles or account for ullage if necessary.

Referring to FIGS. 2 and 3, after obtaining the predetermined nominal maximum dosage, needle 38 is then inserted into a patient (not shown) and plunger 16 is depressed toward front end portion 18 to the end-of-injection position. Upon reaching the end-of-injection position (where some fluid may still be emitting from the needle), plunger 16 is forced further down into housing 12. When this occurs, plunger tip 70 contacts and moves separable retaining member 34, reducing the frictional engagement between needle holder 32 and separable retaining member 34. Further depression of plunger 16 also removes plug member 74 from the opening of retraction cavity 72. When the frictional engagement between needle holder 32 and separable retaining member 34 is less than the retraction force of spring 36, spring 36 rapidly expands, causing needle holder 32 to propel upwardly into the opened retraction cavity 72, simultaneously withdrawing needle 38 at least to a position where needle 38 is withdrawn into housing 12, and neatly tucking end cap 78 into open back end portion 20 of housing 12 to prevent reuse.

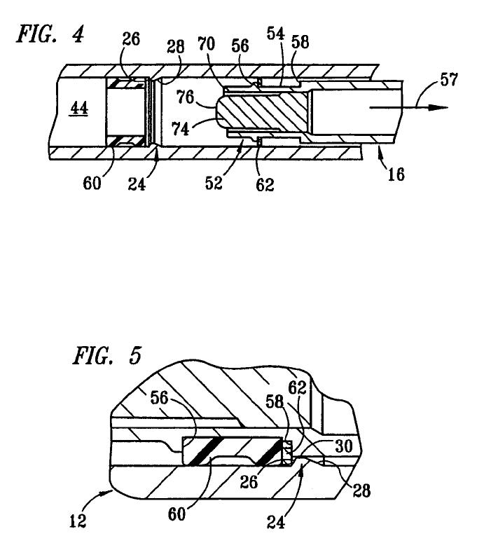

Before needle 38 is inserted into the patient, if the user attempts to move plunger 16 in the rearward direction (marked 57 in FIG. 4) and past the position corresponding to the intended maximum dosage, the user meets resistance. The abutment of stop-ring member 62 with projection 24 causes this resistance. In order to move plunger 16 beyond the position associated with the intended maximum dosage, the user will preferably have to exert about 20 pounds of force on plunger 16. This amount of resistance is generated as a result of the preferred dimensions of stop-ring member 62 and projection 24, and the materials used for each. For example, although the chamfer angle for side 28 is smaller so that stop-ring member 62 can move easily over projection 24 when loading plunger 16 during assembly, the larger chamfer angle of side 26 contributes to the amount of force required to move stop-ring member 62 beyond projection 24. In addition, stop-ring member 62 should be fairly thin but rigid enough that plunger seal 60 is stripped off plunger head 52 if the user pulls plunger 16 beyond the position corresponding to the nominal fixed-dosage. If stop-ring member 62 is too thick or if stop-ring member 62 deforms too easily, when a user attempts to pull plunger 16 beyond the position corresponding to the nominal fixed-dosage, too much pressure may be placed on plunger seal 60 or plunger seal 60 may not resist movement over projection 24, respectively.

As evidenced in FIG. 4, the further withdrawal of plunger head 52 beyond projection 24 will not draw more fluid into syringe 10 because stop-ring member 62 will not move beyond projection 24 without stripping off or removing plunger seal 60 from plunger head 52. Without plunger seal 60, plunger 16 will no longer create suction to draw additional fluid into syringe 10 and the syringe will no longer operate, as plunger seal 60 cannot again be properly mounted on plunger head 52 to create suction. Therefore, removing plunger seal 60 from plunger 16 prevents reuse of syringe 10 even if needle 38 is not retracted after use.

Upon removal from plunger 16, plunger seal 60 preferably remains in housing 12 between inwardly directed projection 24 and front end portion 18. With plunger seal 60 off plunger head 52, stop-ring member 62 preferably remains on and is free to float about plunger head 52.

One skilled in the art will appreciate based on this disclosure that projection 24 could extend further into the housing such that the contact between plunger seal 60 and projection 24 alerts the user that the maximum intended movement of plunger 60 has been reached and if the user attempts to move plunger 16 further rearwardly, plunger seal 60 will be stripped off or removed from plunger 16 without the aid of ring 62.

Other alternations and modifications of the invention will likewise become apparent to those of ordinary skill in the art upon reading the present disclosure, and it is intended that the scope of the invention disclosed herein be limited only by the broadest interpretation of the appended claims to which the inventors are legally entitled.

1. A retractable syringe with a limited maximum usable capacity, comprising:
an elongated hollow syringe housing having a front end portion, a back end portion with an opening, and a longitudinally extending wall between the front end portion and the back end portion;
an elongated plunger disposed for reciprocation in sliding sealed contact with the longitudinally extending wall of the housing, the plunger having a tubular wall defining a front end portion having a head with a plunger seal thereon, a back end portion carrying an end cap, and a hollow interior comprising a retraction cavity;
a retractable needle disposed in the front end portion of the housing, the retractable needle preventing reuse of the syringe following an injection by retracting into the retraction cavity;
a fluid chamber disposed in the housing between the retractable needle and the head of the plunger; and
a dose-limiting structure preventing the plunger from drawing more fluid than the limited maximum usable capacity of the syringe.

2. The retractable syringe of claim 1 wherein the plunger head has a first annular raised shoulder spaced from a second annular raised shoulder by a reduced diameter portion therebetween.

3. The retractable syringe of claim 2 wherein the dose-limiting structure comprises a stop-ring member, the stop-ring member and plunger seal disposed on the reduced diameter portion of the plunger head compressed between the first and second shoulders.

4. The retractable syringe of claim 3 wherein the dose-limiting structure comprises an inwardly directed projection integrally formed on the longitudinally extending wall inside the housing, the projection being spaced from the front end portion of the housing at a location corresponding to the limited maximum usable capacity, wherein the plunger seal is stripped off the plunger head by the force exerted on the plunger seal by the stop-ring member if the plunger is pulled rearwardly beyond the location corresponding to the limited maximum usable capacity.

5. The retractable syringe of claim 4 wherein at the end of aspiration, stop-ring member contacts the projection to alert a user that the limited maximum usable capacity is reached.

6. The retractable syringe of claim 4 wherein the inwardly directed projection has first chamfered side positioned nearer the front end portion of the housing and second chamfered side positioned nearer the back end portion of the housing.

7. The retractable syringe of claim 6 wherein the first chamfered side has a chamfer angle of between about 15 degrees and about 45 degrees.

8. The retractable syringe of claim 6 wherein the first chamfered side has a chamfer angle of about 30 degrees.

9. The retractable syringe of claim 6 wherein the second chamfered side has a chamfer angle of between about 5 degrees and about 10 degrees.

10. The retractable syringe of claim 6 wherein the second chamfered side has a chamfer angle of about 10 degrees.

11. The retractable syringe of claim 3 wherein the stop-ring member is polymeric.

12. The retractable syringe of claim 3 wherein the stop-ring member is metal.

13. The retractable syringe of claim 1 comprising a nominal capacity of 0.5 ml.

14. The retractable syringe of claim 1 wherein the retractable needle is mounted in a retraction mechanism in the front end of the housing with the needle extended in its unretracted position.

15. The retractable syringe of claim 14 wherein the front end portion of the plunger operates the retraction mechanism to retract the needle.

16. The retractable syringe of claim 15 wherein the retraction mechanism comprises a needle holder, separable retaining member and spring, the separable retaining member engages the needle holder with a holding force which exceeds a retraction force applied to the needle holder by the spring when the spring is compressed.

17. The retractable syringe of claim 16 wherein the front end portion of the plunger has a tip aligned to abut against the separable retaining member and slide the retaining member longitudinally relative to the needle holder resulting in the retraction force exceeding the holding force and causing at least part of the retraction mechanism to be retracted into the retraction cavity, to prevent reuse of the syringe.

18. A syringe designed with a limited maximum usable capacity, comprising:
a hollow syringe housing having a first end, a second open end and a longitudinally extending wall between the first and second ends;
a needle disposed in the first end;
a plunger having a front end portion comprising a head having a plunger seal in sliding sealed contact with the housing, and a back end portion with an end cap;
a fluid chamber disposed in the housing between the needle and the head of the plunger; and
a dose-limiting structure preventing the plunger from drawing more fluid than the limited maximum usable capacity of the syringe by stripping the seal off the plunger.

19. The syringe of claim 18 further comprising an apparatus preventing reuse of the syringe following an injection.

20. The syringe of claim 19 wherein the apparatus is a needle retraction mechanism.

21. The syringe of claim 18 wherein the dose-limiting structure is disposed in a fixed location in the housing and is an inwardly facing annular projection.

22. The syringe of claim 18 wherein the plunger head has first annular raised shoulder spaced from a second annular raised shoulder by a reduced diameter portion therebetween.

23. The syringe of claim 22 wherein the dose-limiting structure further comprises a stop-ring member, the plunger seal and stop-ring member disposed on the reduced diameter portion of the plunger head.

24. The syringe of claim 23 wherein the plunger seal is stripped off the plunger when enough force is exerted on the plunger after the stop-ring member has abutted an inwardly directed projection in the longitudinally extending wall of the housing.

25. The syringe of claim 24 wherein the inwardly directed projection has a first chamfered side positioned nearer the first end of the housing and a second chamfered side positioned nearer the second end of the housing.

26. The syringe of claim 25 wherein the first chamfered side has a chamfer angle of between about 15 degrees and about 45 degrees.

27. The syringe of claim 25 wherein the first chamfered side has a chamfer angle of about 30 degrees.

28. The syringe of claim 25 wherein the second chamfered side has a chamfer angle of between about 5 degrees and about 10 degrees.

29. The syringe of claim 25 wherein the second chamfered side has a chamfer angle of about 10 degrees.

30. The syringe of claim 23 wherein the stop-ring member is constructed of metal.

31. The syringe of claim 23 wherein the stop-ring member is constructed of a polymeric material.

32. The syringe of claim 18 wherein the dose-limiting structure is positioned at a location in the housing that is associated with a dose of 0.5 ml.

33. A retractable syringe with a limited maximum usable capacity, comprising:
an elongated hollow syringe housing having a front end portion, a back end portion with an opening, and a longitudinally extending wall between the front end portion and the back end portion;
an elongated plunger disposed for reciprocation in sliding sealed contact with an interior of the housing, the plunger having a tubular wall defining a front end portion having a head with first and second annular raised shoulders separated by a reduced diameter portion therebetween, a back end portion carrying an end cap, and a hollow interior comprising a retraction cavity;
a plunger seal and stop-ring member disposed on the reduced diameter portion of the plunger head compressed between the two shoulders;
a retraction mechanism disposed in the front end portion of the housing, the retraction mechanism being configured for operation by forward movement of the plunger;
an inwardly directed annular projection circumscribing the longitudinally extending wall inside the housing, the inwardly directed annular projection located at a defined point corresponding to the limited maximum usable capacity;
wherein during aspiration, contact between the stop-ring member and the inwardly directed annular projection tactilely signals a user that maximum intended movement of the plunger is reached.

34. The retractable syringe of claim 33 wherein the plunger seal is a releasable plunger seal.

35. The retractable syringe of claim 34 wherein the releasable plunger seal is stripped off the plunger when the plunger is forced past the defined point corresponding to the limited maximum usable capacity.

36. The retractable syringe of claim 33 wherein the inwardly directed annular projection has a first chamfered side located nearer the front end portion of the housing and a second chamfered side located nearer the back end portion of the housing.

37. The retractable syringe of claim 36 wherein the first chamfered side has a chamfer angle of between about 15 degrees and about 45 degrees.

38. The retractable syringe of claim 36 wherein the first chamfered side has a chamfer angle of about 30 degrees.

39. The retractable syringe of claim 36 wherein the second chamfered side has a chamfer angle of between about 5 degrees and about 10 degrees.

40. The retractable syringe of claim 36 wherein the second chamfered side has a chamfer angle of about 10 degrees.

41. The retractable syringe of claim 33 wherein the retraction mechanism comprises a needle holder, separable retaining member and spring, the separable retaining member engages the needle holder with a holding force which exceeds a retraction force applied to the needle holder by the spring when the spring is compressed.

42. The retractable syringe of claim 41 wherein the front end portion of the plunger has a tip aligned to abut against the separable retaining member and slide the retaining member longitudinally relative to the needle holder resulting in the retraction force exceeding the holding force and causing at least part of the retraction mechanism to be retracted into the retraction cavity, to prevent reuse of the syringe.

43. The retractable syringe of claim 42 wherein the retractable needle, needle holder and spring are at least partially retracted into the retraction cavity upon the operation of the retraction mechanism, to prevent reuse of the syringe.

44. The retractable syringe of claim 43 wherein the end cap is lodged into the opening at the back end portion of the housing upon retraction, preventing reuse of the syringe.
